(19) United States
(12) Patent Application Publication
(10) Pub. No.: US 2006/0223965 A1
(43) Pub. Date: Oct. 5, 2006

(54) High strength organic-inorganic hybrid gel materials

(75) Inventor: Roxana Trifu, Shrewsbury, MA (US)

Correspondence Address: ASPEN AEROGELS INC.;IP DEPARTMENT, 30 FORBES ROAD, BLDG. B, NORTHBOROUGH, MA, 01532, US

(73) Assignee: Aspen Aerogels Inc., Northborough, MA (US)

(21) Appl. No.: 11/393,978

(22) Filed: Mar. 30, 2006

Related U.S. Application Data
(60) Provisional application No. 60/594,359, filed on Mar. 31, 2005.

(51) Int. Cl. C07H 1/00; C08G 77/60

(52) U.S. Cl. 528/35; 536/123.1; 528/39

(57) ABSTRACT

Embodiments of the present invention describe hybrid aerogels comprising polymers covalently bonded to an inorganic network and methods for preparing the same. The inorganic network may comprise silica, alumina, titania, zirconia, ceria, yttria or a combination thereof, where silica is the preferred embodiment. The polymers preferably comprise chitosan, pyrrolidine complex of chitosan or any other derivatives of chitosan that are soluble in water, ethanol or combinations thereof.

### CROSS REFERENCE TO RELATED APPLICATIONS

This application claims benefit of priority from U.S. Provisional Patent Application 60/594,359 filed on Mar. 31, 2005 which is hereby incorporated by reference in its entirety as if fully set forth.

### STATEMENT REGARDING FEDERALLY SPONSORED RESEARCH OR DEVELOPMENT

This invention was partially made with Government support under Contract W81XWH-04-C-0046 awarded by the United States Army. The Government may have certain rights in parts of this invention.

### FIELD OF THE INVENTION

This invention pertains to organic-inorganic hybrid gel materials and to methods for preparing the same.

### SUMMARY OF THE INVENTION

Embodiments of the present invention describe gel materials comprising a polysaccharide and an inorganic network, said polysacchride covalently linking two atoms in said inorganic network. A method for preparing said gel materials comprises the steps of: mixing a polysaccharide with an inorganic gel precursor in a suitable solvent; forming a gel from the mixture; and drying the gel. Preferably the polysaccharide has alkoxysilyl groups attached thereto. Another method comprises the steps of: mixing a polysaccharide with an inorganic precursor and a cross-linker in a suitable solvent; forming a gel from the mixture; and drying the gel. Preferably said cross-linker and polysaccharide each comprise at least one isocyanate or isocyanate-reactive group; said cross-linker further comprising an alkoxysilyl group. The isocyanate-reactive groups may comprise OH, COOH, NH₂, NHR or a combination thereof. In general the cross-inker has a formula R₂—Si(OR₁)₃ wherein: OR₁ is an alkoxy group; and R₂ is a functional group capable of forming a covalent bond between the cross-linker and the polysaccharide. Inorganic precursors may be chosen from the general class of metal oxides such as silica. Polysaccharides may comprise sucrose, lactose, maltose, glucose, galactose, fructose, derivatives thereof, or any combination of the preceding. In one embodiment, said polysaccharide comprises chitosan. Once formed, the gels may be dried at supercritical or ambient pressures. The gel materials described exhibit a thermal conductivity of less than about 20 mW/mK, less than about 15 mW/mK or less than about 12 mW/mK. Their densities are between about 0.01 g/cm³ and about 0.5 g/cm³, between about 0.01 g/cm³ and about 0.3 g/cm³, or between about 0.05 and about 0.2 g/cm³. These materials also exhibit improved mechanical properties where the gel material deforms less than about 33%, less than about 20%, less than about 15% or less than about 7% after a compressive loading of about 100 psi for about an hour.

### DESCRIPTION

Aerogel materials find one common use as thermal insulation. Due to their highly porous structure and ultra fine pore size, aerogels have extremely low thermal conductivity coefficients. Though considered as one of the best thermal insulating materials, their use has been limited to a large extent by their low mechanical strength, most notably low compression strength.

At reduced pressures (e.g. vacuum) aerogels typically exhibit larger R-values (resistance to heat transfer) due to elimination of gas conduction through the pores. About a 4-fold increase of the R-value per inch is often noted when an aerogel is evacuated at pressures below 1 torr. Accordingly, aerogels with improved compression resistance would better resist potential densification during evacuation thereby maximizing R-value due to lower solid conduction.

Ambient pressure applications of aerogels also derive benefit from enhanced mechanical strength. For example, although increasing the aerogel's density generally results in higher mechanical strength, it is typically at the cost of significantly increasing the thermal conductivity and therefore is not a suitable approach. Hence it is also desired that that improvements in mechanical strength of aerogels should not adversely affect thermal properties of the same.

Various properties of aerogels, such as: mechanical, thermal, optical, acoustic etc., depend heavily on the preparation methods. The insulation performance of aerogels can be very sensitive to density. For instance the optimal thermal insulation performance for silica aerogels is typically within the density range of about 0.05 and about 0.20 g/cc. Above this density range, significantly higher thermal conductivities result.

Within the context of embodiments of the present invention “aerogels” or “aerogel materials” along with their respective singular forms, refer to gels containing air as a dispersion medium in a broad sense, and include gels processed via supercritical drying in a narrow sense.

Production of aerogels typically involves replacing the liquid solvent phase within the pores of a wet gel by air, preferably without allowing substantial collapse of the pore structure. The sol-gel process is one method for preparing wet gels, where upon drying can result in aerogels. Sol-gel process is described in detail in Brinker C. J., and Scherer G. W., Sol-Gel Science; New York: Academic Press, 1990; hereby incorporated by reference. For example, a wet silica gel is typically prepared through the sol-gel process, which involves the formation of a sol through hydrolysis of a silica precursor, and the subsequent gelling through condensation between the species evolved from the hydrolysis. The resultant gel is frequently subject to a post-gelling process, which may involve aging, solvent exchange, and any additional chemical modifications. The sol-gel process may be considered the most important step in aerogel preparation because the aerogel properties are to a large extent determined by this step. Various chemical modifications can also be carried out during this step in order to improve the properties of the resultant aerogels. For instance, hybrid organic-inorganic aerogels can be prepared using sol-gel synthesis, where such hybrid exhibits improved properties over the inorganic-only counterpart.

To date, efforts to incorporate organic modifiers into silica aerogels to form hybrid aerogels with improved properties have been made. For instance, published U.S. Patent Application No. 20040132846 teaches a method of making silica aerogel monoliths wherein the strength has been improved through cross-linking the preformed silica gels with a cross-linking agent, such as poly(hexamethylene diisocyanate). However, this approach also significantly increases the density of the resultant aerogels. In addition, this approach involves a lengthy post-gelling solvent exchange process and a high temperature, prolonged post-exchange reaction process.

U.S. Pat. No. 6,825,260 describes preparation of aerogel-like materials composed of nanoporous, interpenetrating organic-inorganic networks. The effect of the polymers on the aerogel strength is not described in this disclosure. Although aerogels with improved mechanical properties are reported, the improved mechanical properties may be a consequence of doping with macroscopic reinforcing fibers. Further, the aerogels reported therein have thermal conductivity coefficients in the range of 34-50 mW/m-K, significantly higher than that of the aerogels of the present invention.

Chung, Y. et al (Material Research Society Symposium Proceedings, Vol. 180, P. 981, 1990) reported the synthesis of organically modified silicate materials with rubbery elasticity by reacting common silicon alkoxides with polydimethylsiloxane through the sol-gel process. The resulting product was composed of covalently bonded organic polymer and inorganic silicic groups. Although this work represents a useful approach to making organic-inorganic hybrid material with improved mechanical properties, it is limited to high density, low porosity materials that are fundamentally different from aerogels. The amounts of the organic component in the hybrid material are relatively large, which prohibits the formation of a low density, aerogel-like material.

Natural materials are attractive candidates for silica aerogel modification due to their potential low cost and environmentally friendly nature. Studies on chemical modification of aerogels with natural polymers or their derivatives are also known. For example, silica-chitosan aerogels with novel functionalities and reduced linear shrinkage compared to pure silica aerogels have been reported (U.S. Pat. No. 6,303,046; M. R. Ayers, A. J. Hunt, J. Non-Cryst. Solids 285 (2001)123-127). The preceding references involve a number of limitations. For example, the wet gel prepared in these approaches requires extensive solvent exchange to remove the water before the supercritical extraction and the processing is time consuming and can be as long as over a week. More importantly, the main interaction between the organic component and the inorganic component in these silica-chitosan hybrid aerogels are hydrogen bonds. Hydrogen bonds are relatively weak, typically less than about 5% the strength of covalent bonds. Accordingly, it is likely that only very limited improvement in mechanical strength may result from the introduction of chitosan in such methods. It is desirable to connect chitosan through covalent bonding to the silica to improve its reinforcement to the silica network. There is no covalent bonding formed in the above reported approaches. In sum, there remains a need for aerogels and related materials having improved mechanical properties and related processing techniques without the aforementioned limitations.

In one aspect, the present invention involves hybrid organic-inorganic gel materials comprising a polysaccharide covalently bonded to an inorganic network. The inorganic network may comprise a metal oxide such as silica, titania, zirconia, alumina, hafnia, yttria, ceria or combinations thereof; where silica is utilized in the preferred embodiments. Polysaccharides may be regarded as polymers comprising repeat units of monosaccharides (or disaccharides or both) where the general formula of the polymer is Cₙ(H₂O)ₙ₋₁. Examples of polysaccharides include but are not limited to: starches, glycogens, cellulose, chitosan, acidic polysaccharides and bacterial capsule polysaccharides. Polysaccharides may be homo- or hetero polymers and based on mono- and/or disaccharides including but are not limited to: sucrose, lactose, maltose, glucose, galactose, fructose, their derivatives and combinations thereof.

In a further aspect of the present invention, the polysaccharide covalently links at least two atoms in the inorganic network. Stated differently, at least two atoms in the inorganic network are covalently linked through a polysaccharide. In an even further aspect, the polysaccharide is bonded through a cross-linkage to the inorganic network where said cross-linkage preferably comprises a urethane (carbamate) or urea functional group. In an embodiment, a cross-linker is used to create the cross-inkage between the polysaccharide and the inorganic network. Preferably said cross-linker comprises a hydrolysable alkoxy group suitable for sol-gel chemistry and at least one isocyanate or an isocyanate-reactive group such as OH, COOH, NH₂, NHR or a combination thereof. A general formula for such cross-linkers is R₂—Si(OR₁)₃; Wherein OR₁— is a generic hydrolysable alkoxy group which may be cleaved from said cross-linker to form a covalent bond between the cross-linker and the inorganic network, and R₂ is a functional group capable of forming covalent bond between the cross-linker and the polysaccharide. In an embodiment, R₂ comprises an isocyanate or an isocyanate-reactive group. Accordingly, hydrolysis and condensation of the alkoxy group links the cross-linker and attachments thereto (e.g. polysaccharide), to the inorganic network. Said polysaccharide is preferably functionalized with isocyanates or isocyanate reactive groups such as OH, COOH, NH₂, NHR or a combination thereof. Preferably the polysaccharide is soluble in water, ethanol or both. The choice of functional groups for the polysaccharide and the cross-linker therefore should be such that a reaction between the two results in a covalent bond therebetween; preferably reaction between the two results in formation of a urea or urethane group. Such reactions, particularly those resulting in a urethane group are described in U.S. Pat. No. 5,990,184 hereby incorporated by reference.

Aminated polysaccharides can react with an isocyanate group of a cross-linker, resulting in covalent linkage comprising a urea group. Aminated polysaccharides may be naturally occurring or modified as such. Examples of aminated polysaccharides suitable for use in the instant invention and methods for their preparation are described in U.S. Pat. Nos. 3,472,840 and 3,431,254 which are hereby incorporated by reference.

In hybrid gel materials of the preferred embodiment, the polysaccharides comprise chitosan and the inorganic network comprises silica. The cross-inkage may comprise a urea or urethane group, more preferably urea group. Accordingly, a cross-inker for forming said cross linkage comprises an isocyanate and an alkoxysilyl group. Subsequent gellation of the polysaccharide (e.g. chitosan) with the silica precursor leads to a strong covalent bond therebetween, and therefore a strong resultant gel. As used herein “chitosan” refers to polymers comprising chitosan, pyrrolidine complex of chitosan or any other derivatives of chitosan. Once gelled, these hybrid materials may be dried in a variety of ways to form an aerogel, xerogel, or cryogel. In the preferred embodiments, the gel materials are dried to obtain an aerogel.

Hybrid aerogels according to embodiments of the present invention demonstrate significantly improved mechanical strength, particularly resilience, when compared to typical inorganic aerogels (e.g. silica aerogel.) The improved strength is believed to result from the introduction of the polysaccharide into the inorganic network. Furthermore, introduction of the polymer does not adversely affect the thermal conductivity of the resultant aerogels. The thermal conductivity coefficients may range from about 10 to about 35 mW/m-K and preferably in the range of about 10 to about 15 mW/m-K. The densities may range between about 0.01 g/cm³ and about 0.5 g/cm³, between about 0.01 g/cm³ and about 0.3 g/cm³ or between about 0.05 and about 0.2 g/cm³.

In embodiments of the present invention sol-gel chemisty is used to prepare the hybrid gel materials. These gel materials may be regarded as a three-dimensionally linked polymeric structure. Generally, under certain conditions such as adequate dilution and/or acidic media, polysaccharides such as chitosan become miscible in aqueous and alcoholic solutions, and can be mixed into a metal oxide sol (e.g. silica sol), during the sol-gel process. Hydrolysis and condensation of the polymer and the metal oxide (e.g. siliceous) species in the sol-gel lead to the formation of strong linkages that covalently bond the polymer to the silica network. In the resultant aerogels, the polymer is integrated in to the silica matrix on the nanometer level and it provides substantial reinforcement to the silica network without compromising the aerogel's thermal insulation properties. The aerogel can optionally be reinforced with a fibrous structure to further improve its mechanical strength, handling and flexibility.

Preferably the polysaccharides comprise an isocyanate or an isocyanate-reactive group such as OH, COOH, NH₂, NHR or a combination thereof. The more preferred polysaccharides comprises chitosan, pyrrolidine complex of chitosan or any other derivatives of chitosan, where such compounds are preferably soluble in water, ethanol or both. Further, the polysaccharide can have an average molecular weight ranging from about 1000 to about 2,000,000 and preferably from about 5,000 to about 1000,000. The weight percentage of the polysaccharide in the gel material may range from about 0.5% to about 50%, and preferably from about 2% to about 10%.

The polysaccharide and the silica may be covalently bonded through a cross-inker. Said cross-inker may be represented with the general formula (R₁—O)₃Si—R₂, wherein R₁—O is a generic hydrolysable group which may be cleaved from said cross-linker to form a covalent bond between the cross-linker and the inorganic network network, and wherein R₂ is a functional group capable of forming a urea or urethane linkage between the cross-linker and the amine groups on the polysacchride backbone. Preferably, R₁ comprises an ethyl group. Also preferably, R₂ comprises an isocyanate group. Most preferably, the cross-linker is isocyanatopropyl triethoxysilane (OCN—(CH₂)₃—Si(OC₂H₅)₃).

Aerogel materials can be further strengthened by incorporating a fibrous structure therein. For instance the pre-gel mixture may be introduced into a fibrous structure where upon gellation and subsequent drying yields a fiber-reinforced aerogel. Fibers suitable for reinforcement of aerogel materials may comprise organic polymer-based fibers (e.g. polyethylenes, polypropylenes, polyacrylonitriles, polyamids, aramids, polyesters etc.) inorganic fibers (e.g. carbon, quartz, glass, etc.) or both and in forms of, wovens, non-wovens, mats, felts, battings, lofty battings, chopped fibers, or a combination thereof. Aerogel composites reinforced with a fibrous batting, herein referred to as “blankets”, are particularly useful for applications requiring flexibility since they can conform to three-dimensional surfaces and provide very low thermal conductivity. Aerogel blankets and similar fiber-reinforced aerogel composites are described in published US patent application 2002/0094426A1 and U.S. Pat. Nos. 6,068,882, 5,789,075, 5,306,555, 6,887,563, and 6,080,475, all hereby incorporated by reference, in their entirety.

In one embodiment, preparation of gel materials comprises mixing a polysaccharide with a cross-linker and an inorganic precursor in a suitable solvent, said polysaccharide and cross-linker each comprising at least one isocyanate or isocyanate-reactive group such as OH, COOH, NH₂, NHR wherein a reaction between said cross-linker and polysaccharide results in a covalent linkage therebetween; optionally incorporating an additive in the mixture; optionally introducing the reaction mixture into a fibrous structure; forming a gel from the mixture; optionally aging the gel; and drying the gel.

In another embodiment, preparation of gel materials comprises mixing a polysaccharide with a cross-linker before mixing with a metal oxide precursor in a suitable solvent, said polysaccharide and cross-linker each comprising at least one isocyanate or an isocyanate-reactive group such as OH, COOH, NH₂, NHR wherein a reaction between said cross-inker and polysaccharide results in a covalent linkage therebetween; optionally incorporating an additive in the mixture; optionally introducing the reaction mixture into a fibrous structure; forming a gel from the mixture; optionally aging the gel; and drying the gel.

In another embodiment, preparation of gel materials comprises the steps of: mixing a polysaccharide and an inorganic precursor in a suitable solvent; said polysaccharide comprising hydrolysable alkoxy groups; optionally incorporating an additive in the mixture; optionally introducing the reaction mixture into a fibrous structure; forming a gel from the mixture; optionally aging the gel; and drying the gel.

In a specific embodiment, the preparation of gel materials comprises the steps of: mixing an amount of a polysaccharide comprising chitosan with an amount of an inorganic precursor comprising silica and an alkoxylsilyl-containing cross-linker in a solvent; optionally incorporating an additive in the mixture; optionally introducing the reaction mixture into a fibrous structure; forming a gel from the mixture; optionally aging the gel; and drying the gel.

In another specific embodiment, the preparation of gel materials comprises the steps of: mixing an amount of a polysaccharide comprising chitosan with an amount of an inorganic precursor comprising silica in a suitable solvent wherein said chitosan comprises at least one hydrolysable alkoxy functional group; optionally incorporating an additive in the mixture; optionally introducing the reaction mixture into a fibrous structure; forming a gel from the mixture; optionally aging the gel; and drying the gel.

In general, inorganic precursors such as metal oxides are suitable for use provided that they can participate in the sol-gel process therby forming an inorganic network. The silica precursors used in some methods may be chosen from but are not limited to: alkoxysilane, partially hydrolyzed alkoxysilanes, tetraethoxylsilane(TEOS), partially hydrolyzed TEOS, condensed polymers of TEOS, tetramethoxylsilane (TMOS), partially hydrolyzed TMOS, condensed polymers of TMOS, tetra-n-propoxysilane, partially hydrolyzed and/or condensed polymers of tetra-n-propoxysilane, or combinations thereof. TEOS, partially hydrolyzed polyethysilicates, and polyethylsilicates are some of the more common commercially available silica precursors.

Gels may be dried in a variety of ways as known in the art such as but not limited to ambient pressure drying and supercritical drying. U.S. Pat. No. 6,670,402 herein incorporated by reference, teaches drying via rapid solvent exchange of solvent(s) inside wet gels using supercritical CO₂ by injecting supercritical, rather than liquid, CO₂ into an extractor that has been pre-heated and pre-pressurized to substantially supercritical conditions or above to produce aerogels. U.S. Pat. No. 5,962,539 herein incorporated by reference, describes a process for obtaining an aerogel from a polymeric material that is in the form a sol-gel in an organic solvent, by exchanging the organic solvent for a fluid having a critical temperature below a temperature of polymer decomposition, and supercritically drying the fluid/sol-gel. U.S. Pat. No. 6,315,971 herein incorporated by reference, discloses processes for producing gel compositions comprising: drying a wet gel comprising gel solids and a drying agent to remove the drying agent under drying conditions sufficient to minimize shrinkage of the gel during drying. Also, U.S. Pat. No. 5,420,168 herein incorporated by reference describes a process whereby Resorcinol/Formaldehyde aerogels can be manufactured using a simple air drying procedure. Finally, U.S. Pat. No. 5,565,142 herein incorporated by reference describes subcritical drying techniques. The embodiments of the present invention may be practiced with these drying techniques. In some embodiments, it is preferred that the drying is performed at vacuum to below super-critical pressures (pressures below the critical pressure of the fluid present in the gel at some point) and optionally using surface modifying agents.

Generally gels may be formed via maintaining the mixture in a quiescent state for a sufficient period of time, changing the pH of the solution, directing a form of energy onto the mixture, or a combination thereof. Exemplary forms of energy include: a controlled flux of electromagnetic (ultraviolet, visible, infrared, microwave), acoustic (ultrasound), or particle radiation.

In all of the above methods, another optional step may be included wherein additives are incorporated into the aerogel material. Preferably said additives are included in the mixture prior to gellation thereof. Examples of additives include strengthening fibers, fillers, particulates and opacifiers. Opacifiers are further exemplified by, but not limited to: B₄C, Diatomite, Manganese ferrite, MnO, NiO, SnO, Ag₂O, Bi₂O₃, TiC, WC, carbon black, titanium oxide, iron titanium oxide, zirconium silicate, zirconium oxide, iron (I) oxide, iron (III) oxide, manganese dioxide, iron titanium oxide (ilmenite), chromium oxide, silicon carbide or mixtures thereof. Furthermore, these methods may also involve aging steps, addition of catalysts (for catalyzing gellation) as regularly practiced in the art.

The methods provided herein can be used to prepare a continuous monolith, discrete particulate, composite or other forms of aerogels. These and still further embodiments of the present invention are described in greater detail below.

### DETAILED DESCRIPTION USING A PREFERRED EMBODIMENT

Preparation of gel materials are discussed in greater detail by way of a specific preferred embodiment utilizing polysaccharides which comprise chitosan and inorganic precursors which comprise silica. This discussion should only serve to aid in understanding aspects of the instant invention and therefore may not serve to limit the scope or spirit of the present invention in any manner.

Certain polysaccharides with functional groups such as amine (NH, NHR, etc.) and hydroxyl (OH) groups are particularly suitable candidates as polysaccharides for hybrid gel production. These preferred polysaccharides can be dissolved in the sol-gel solution, typically aqueous and alcoholic, to form a homogeneous sol and subsequent gel with the other sol-gel components (i.e. inorganic precursors.) More importantly, the functional groups on the polysaccharides can form covalent bonding to the inorganic network such that it affords significant mechanical reinforcement to the resultant gels, and particularly aerogels. In addition, introduction of these polysaccharides does not deleteriously affect the thermal conductivity coefficient, density, and other important properties of the base inorganic aerogel.

A preferred class of polysaccharides is chitosan or its derivatives. Chitosan is a commercially available material derived from chitin and one of the most abundant organic compounds on earth. Chitosan is derived by the base-hydrolysis of chitin to effect the removal of acyl groups from its acylamine substituents. In its fully deacylated form, chitosan is a polymer containing beta-(1-4)-2-amino-2-deoxy-D-glucose units. A particularly preferred polymer in this invention is a derivative of chitosan Kytamer™ PC. It has been discovered that the pyrrolidine complex of chitosan is superior to unmodified chitosan in both solubility and compatibility with the silica/ethanol system.

The preparation method for this new type of hybrid chitosan-silica gel must also be selected carefully so that the desired effects of the polysaccharide on the silica can be realized without compromising the advantageous features of pure silica aerogels such as low thermal conductivity coefficient.

It was first discovered that by using a suitable amount of R—Si(OR)₃ type cross-linker along with a Si(OR)₄ or other appropriate silica precursors during the initial gel formation, the polysaccharide can be effectively introduced into the inorganic network with good chemical linkage but without: a) major solubility issues, b) high density gel formation, and c) lengthy required processing steps. In a more preferred embodiment, the R₂—Si(OR₁)₃ type precursor is isocyanatopropyl triethoxysilane (OCN—(CH₂)₃—Si(OC₂H₅)₃). The isocyanate functional group reacts with the amine groups on chitosan to form a urea linkage, while the other end of the cross-linker, the alkoxy group, covalently bonds the chitosan onto the silica network. The reaction scheme is illustrated in FIG. 1.

Additional reinforcing components can also be optionally added to improve the mechanical strength of the hybrid gels or aerogels. In particular, macroscopic fibers in the form of nonwovens or mats, for example, can be added. Additionally, nanoparticulate material such as silica nanoparticles as additives can be added as the reinforcing material.

It is also noted that the resultant hybrid aerogels show significantly improved compression resistance over the analogous silica aerogels while preserving the superior thermal insulation performance and low densities of the silica aerogels.

Several formulations of final density in the range of 0.09-0.11 g/cc with different concentrations of chitosan cross-inked with isocyanatopropyl triethoxysilane have been prepared and processed under various conditions. The properties of the resulting hybrid aerogel composites (reinforced with polyester batting) have been evaluated based on the method below. The corresponding data is tabulated in Table 1.

The total deformation Dt was calculated based on initial thickness (tᵢ) and the final one (tᵣ) measured 1 h after compression. This is a measure of compression resistance and resilience of aerogel composites.
Dt(%)=(tᵢ−tᵣ)/tᵢ×100

Thermal conductivities at ambient of 12 mW/mK and densities of 0.1 g/cc and below have been obtained for the hybrid aerogel composites. Lighter hybrid aerogel composites can be obtained with increasing chitosan concentration and with soaking the gel in basic bath prior to the hydrophobicity treatment. It is important to note that thermal performance was not adversely affected by the polymer doping. The silica composite control (0% chitosan) tested 12.9 mW/mK, while its counterpart with 4% chitosan aged under same conditions showed 12.4 mW/mK. It is quite possible to optimize the formulation and processing conditions to achieve thermal conductivities much lower than what was achieved through the representative examples.

TABLE 1

| | Thermal | | |
|---|---|---|---|
| Chitosan | Conductivity at | | Total |
| Doping | 100 F., 760 torr | Density | Deformation |
| (%) | (mW/mK) | (g/cc) | (%) |
| 0 | 12.9 | 0.104 | 32.2 |
| 1 | 11.9 | 0.100 | 19.2 |
| 2 | 13.7 | 0.097 | 7.2 |
| 3 | 13.2 | 0.094 | 14.5 |
| 4 | 12.4 | 0.093 | 6.9 |

### EXAMPLE 1

Chitosan (Mw 600,000 from Fluka) 0.4 g was dissolved in 50 mL water in the presence of 0.5 mL acetic acid. Isocyanatopropyl triethoxysilane 0.47 g was added dropwise to the chitosan solution. Prepolymerized ethyl silicate 100 mL and 180 mL of ethanol were slowly added to the cross-linked chitosan solution and stirring was continued for another hour at room temperature. Ammonium hydroxide (30% in water) 0.8 g in 2 mL ethanol was added to the reaction mixture. The sol was cast into 5″×5″ molds while infused with polyester fiber or fiber battings. Gelation occurred in 5 minutes, and the gels were soaked in ammonia ethanolic solution for 4 hours at room temperature. The bath was exchanged with hexamethyldisilazane ethanolic solution and the gels were heated at 55° C. for 2 days. Extraction of the alcohol was accomplished by supercritical CO₂ drying at 1500 psi and 55° C. in 4 hours.

### EXAMPLE 2

Chitosonium pyrrolidine carboxylate (Amerchol) 0.58 g was dissolved in 50 mL water. Ethanol 40 mL was added over the chitosan solution and the mixture was stirred for 15 minutes. Prepolymerized ethyl silicate 95 mL was mixed with 142 mL ethanol and 1 mL isocyanatopropyl triethoxysilane. The chitosan solution was slowly added to the silica sol containing the cross-inker and the mixture was stirred at room temperature for 30 minutes. Ammonium hydroxide 1.2 g in 20 mL ethanol was added dropwise to the reaction mixture and the hybrid sol was prepared as monolith and on fiber reinforcement. The gelation time was 6 minutes. The gels were sealed in molds and left undisturbed for 1 hour, followed by treatment with basic ethanolic solution in turn followed by hydrophobicity treatment at 60° C. Supercritical extraction of the gels resulted in white opaque hybrid aerogels with no cracks.

### DESCRIPTION OF FIGURES

FIG. 1 Depicts one of many possible reactions for formation of a chitosan-silica hybrid gel material.

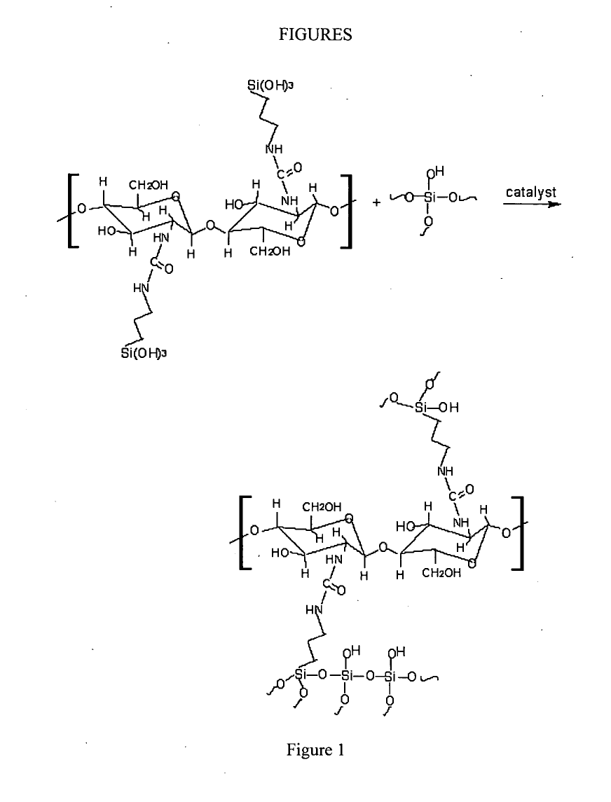

FIG. 2 Shows a plot of compression resistance as a function of chitosan loading in a chitosan-silica hybrid aerogel composite.

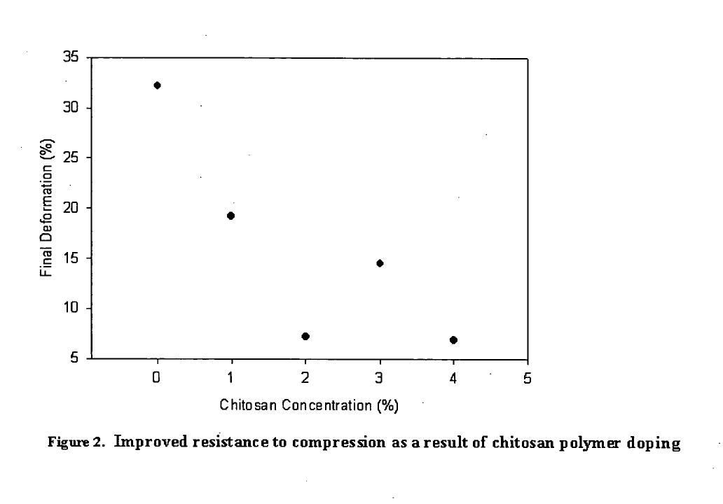

What is claimed is:

1. A gel material comprising a polysaccharide and an inorganic network, said polysacchride covalently linking at least two atoms in said inorganic network.

2. The gel material of claim 1 wherein the inorganic network comprises a metal oxide.

3. The gel material of claim 2 wherein the inorganic network comprises silica.

4. The gel material of claim 1 wherein the polysaccharide comprises sucrose, lactose, maltose, glucose, galactose, fructose, derivatives thereof, or any combination of the preceding.

5. The gel material of claim 1 wherein the polysaccharide comprises chitosan.

6. The gel material of claim 1 wherein the covalent linkage comprises a urea or urethane link.

7. The gel material of claim 1 wherein the gel material is an aerogel.

8. The gel material of claim 1 wherein the gel material is a xerogel.

9. The gel material of claim 1 further comprising a fibrous structure.

10. The gel material of claim 9 wherein the fibrous structure comprises: felt, mat, batting, lofty batting or a combination thereof.

11. The gel material of claim 1 having a thermal conductivity of less than about 20 mW/mK, less than about 15 mW/mK or less than about 12 mW/mK.

12. The gel material of claim 1 having a density between about 0.01 g/cm³ and about 0.5 g/cm³, between about 0.01 g/cm³ and about 0.3 g/cm³ or between about 0.05 and about 0.2 g/cm³.

13. The gel material of claim 1, wherein the gel material deforms less than about 33%, less than about 20%, less than about 15% or less than about 7% after a compressive loading of about 100 psi for about an hour.

14. A method for preparing a gel material comprising the steps of:
mixing a polysaccharide with an inorganic gel precursor in a suitable solvent wherein said polysaccharide comprises alkoxysilyl groups;
forming a gel from the mixture; and
drying the gel.

15. The method of claim 14 wherein the inorganic precursor comprises a metal oxide.

16. The method of claim 15 wherein the inorganic precursor comprises silica.

17. The method of claim 14 wherein the polysaccharide comprises sucrose, lactose, maltose, glucose, galactose, fructose, derivatives thereof, or any combination of the preceding.

18. The method of claim 14 wherein the polysaccharide comprises chitosan.

19. The method of claim 14 wherein the gel is dried using a supercritical fluid.

20. The method 1 of claim 14 wherein the gel is dried at ambient pressures.

21. The method of claim 14 further comprising the step of adding an additive to the mixture.

22. The method of claim 14 further comprising the step of introducing the mixture into a fibrous structure.

23. The method of claim 14 further comprising the step of aging the gel.

24. The method of claim 14 wherein the mixture is formed into a gel by changing the pH thereof.

25. The method of claim 21 wherein the additive comprises, opacifiers, chopped fibers, particulates or a combination thereof.

26. The method of claim 22 wherein the fibrous structure is a felt, mat, batting or a combination thereof.

27. The method of claim 23 wherein aging is carried out at elevated temperatures.

28. The method of claim 14 wherein the gel material has a thermal conductivity of less than about 20 mW/mK, less than about 15 mW/mK or less than about 12 mW/mK.

29. The method of claim 14 wherein the gel material has a density between about 0.01 g/cm³ and about 0.5 g/cm³, between about 0.01 g/cm³ and about 0.3 g/cm³, or between about 0.05 and about 0.2 g/cm³.

30. The method of claim 14, wherein the gel material deforms less than about 33%, less than about 20%, less than about 15% or less than about 7% after a compressive loading of about 100 psi for about an hour.

31. A gel material according to claim 14.

32. A method for preparing a gel material comprising the steps of:
mixing a polysaccharide with an inorganic precursor and a cross-linker in a suitable solvent, said cross-linker and polysaccharide each comprising at least one isocyanate or isocyanate-reactive group;
forming a gel from the mixture; and
drying the gel.

33. The method of claim 32 wherein the cross-inker further comprises an alkoxysilyl group.

34. The method of claim 32 wherein the isocyanate-reactive group comprises OH, COOH, NH₂, NHR or a combination thereof

35. The method of claim 34 wherein the cross-linker has a general formula R₂—Si(OR₁)₃ wherein: OR₁ is an alkoxy group; and
R₂ is a functional group capable of forming covalent bond between the cross-linker and the polysaccharide.

36. The method of claim 32 wherein the inorganic precursor comprises a metal oxide.

37. The method of claim 32 wherein the inorganic precursor comprises silica.

38. The method of claim 32 wherein the polysaccharide comprises sucrose, lactose, maltose, glucose, galactose, fructose, derivatives thereof, or any combination of the preceding.

39. The method of claim 32 wherein the polysaccharide comprises chitosan.

40. The method of claim 32 wherein the gel is dried using a supercritical fluid.

41. The method 1 of claim 32 wherein the gel is dried at ambient pressures.

42. The method 1 of claim 32 wherein the gel material has a thermal conductivity of less than about 20 mW/mK, less than about 15 mW/mK or less than about 12 mW/mK.

43. The method of claim 32 wherein the gel material has a density between about 0.01 g/cm³ and about 0.5 g/cm³, between about 0.01 g/cm³ and about 0.3 g/cm³, or between about 0.05 and about 0.2 g/cm³.

44. The method of claim 32 wherein the gel material deforms less than about 33%, less than about 20%, less than about 15% or less than about 7% after a compressive loading of about 100 psi for about an hour.

45. A gel material according to claim 32.
